(19) United States
(12) Patent Application Publication
(10) Pub. No.: US 2006/0034898 A1
(43) Pub. Date: Feb. 16, 2006

(54) Method for using FIFRA-exempt substances, materials exhibiting antimicrobial, insecticidal, biocidal and/or odor controlling properties having one or more of these substances coated thereon and/or incorporated therein, and products that are made using these materials

(76) Inventors: James A. Amodt, Sisters, OR (US); John W. Colvin, Canby, OR (US); Larry O. Taylor, Nipomo, CA (US)

Correspondence Address: HOLLAND & BONZAGNI, P.C., 171 DWIGHT ROAD, SUITE 302, LONGMEADOW, MA, 01106-1700, US

(21) Appl. No.: 11/200,589

(22) Filed: Aug. 9, 2005

Related U.S. Application Data
(60) Provisional application No. 60/600,462, filed on Aug. 10, 2004.

(51) Int. Cl. A61K 36/899; A61K 36/534; A61K 36/14; A61K 9/70

(52) U.S. Cl. 424/443; 424/747; 424/750; 424/770

(57) ABSTRACT

A method for using FIFRA-exempt substances (e.g., essential oils), materials having one or more such substances coated thereon and/or incorporated therein, and products that are made using these materials, are provided. The properties demonstrated by the inventive materials and products include the control or killing of, for example, decay causing organisms, human pathogens, insects, nematodes, bivalves and spats, and odor removal by destruction of bacteria, mold, fungus and yeasts.

### RELATED APPLICATION

This application claims priority from U.S. Provisional Patent Application Ser. No. 60/600,462, filed Aug. 10, 2004.

### TECHNICAL FIELD OF THE INVENTION

The present invention basically relates to a method for using FIFRA-exempt substances (e.g., essential oils), materials exhibiting antimicrobial, insecticidal, biocidal and/or odor controlling properties having one or more such substances coated thereon and/or incorporated therein, and products that are made using these materials.

### BACKGROUND AND SUMMARY OF THE INVENTION

The Federal Insecticide, Fungicide, and Rodenticide Act (FIFRA), which controls pesticide distribution, sale and use, requires that manufacturers register each pesticide and its label with the Environmental Protection Agency (EPA) before it can be manufactured for commercial use. A “pesticide” is defined in Section 2(u) of FIFRA as “(1) any substance or mixture of substances intended for preventing, destroying, repelling, or mitigating any pest, (2) any substance or mixture of substances intended for use as a plant regulator, defoliant, or desiccant, and (3) any nitrogen stabilizer.”

In 1996, EPA exempted certain minimum risk pesticides from FIFRA requirements if they satisfy certain conditions. The exemption provision is located in § 152.25(g) of Title 40 of the Code of Federal Regulations. To qualify for an exemption as a minimum risk pesticide, each active ingredient in the pesticide product must be listed in 40 C.F.R. § 152.25(g)(1). Currently, this list contains more than 30 active ingredients, including essential oils such as cedar oil and mint or mint oil.

Cedar or cedarwood oil is often used in mothproofing, and may contribute to the control of certain other insects. Tests involving various forms of cedar (e.g., cedar chips, cedar oil, and sachet bags of cedar shavings) indicate that cedar works best in confined spaces such as clothes storage bins, but has little effect in other applications.

The essential oil of mint has been used to control certain pests on plants, reducing the number of eggs laid and the amount of feeding damage by certain insects.

Incorporating oils into plastic materials has been deemed undesirable where such materials are not generally deemed acceptable for injection molding or extrusion. Segregation within the molded or extruded polymer causes poor dimensional control, while poorly formed and non-uniform surface finish generally requires additional finishing. Moreover, the rate of expulsion of the oil from these materials varies greatly, depending in part, upon the concentration of oil within the composition, the use temperature, and other manufacturing and operational factors. As such, in practice, it is quite difficult to manufacture a standardized product.

The present invention dispels preconceived notions regarding the viability of applying oils to and/or incorporating oils within a material, and basically provides a method for using FIFRA-exempt substances such as essential oils (e.g., cedarwood oil, mint oil) to make materials and products exhibiting antimicrobial, insecticidal, biocidal and/or odor controlling properties.

The present invention also provides the above-referenced materials which exhibit at least one of the noted properties and which have one or more FIFRA-exempt substances coated thereon and/or incorporated therein.

The present invention further provides products that are made using these materials.

In one such embodiment, the inventive product is a flexible, protective glove having an inside surface that is coated with one or more FIFRA-exempt substances.

In another such embodiment, the inventive product is a row cover that shields plants from frost, while protecting the plants from insects, and the bacteria, molds and fungus in the soil. The row cover is prepared using a plastic sheet material that is coated and/or impregnated with one or more FIFRA-exempt substances.

In yet another such embodiment, the inventive product is a painted item having at least one surface covered by a paint or paint formulation having one or more FIFRA-exempt substances incorporated therein.

In yet another such embodiment, the inventive product is prepared from (or coated with) a wax-based composition having one or more FIFRA-exempt substances incorporated therein.

Other features and advantages of the invention will be apparent to one of ordinary skill from the following detailed description.

Unless otherwise defined, all technical and scientific terms used herein have the same meaning as commonly understood by one of ordinary skill in the art to which this invention belongs. All publications, patent applications, patents and other references mentioned herein are incorporated by reference in their entirety. In case of conflict, the present specification, including definitions, will control. In addition, the materials, methods, and examples are illustrative only and not intended to be limiting.

### BEST MODE FOR CARRYING OUT THE INVENTION

By way of the subject invention, the present inventors have made the surprising discovery that FIFRA-exempt substances such as essential oils may be effectively applied to and/or incorporated within a material, supplying that material with antimicrobial, insecticidal, biocidal, and/or odor controlling properties.

The properties demonstrated by the inventive materials (as well as products prepared from or using these materials) include the control or killing of, for example, decay causing organisms, human pathogens, insects, nematodes, bivalves and spats, and odor removal by destruction of bacteria, mold, fungus and yeasts.

The substances used to provide these antimicrobial, insecticidal, biocidal, and/or odor controlling properties, which are listed in 40 C.F.R. § 152.25(g)(1), do not require EPA registration as pesticides. Moreover, these substances have been designated GRAS (i.e., Generally Regarded As Safe) by the Food and Drug Administration (FDA). In other words, these substances have been tested and deemed acceptable for human use by the FDA.

Substances Listed in 40 C.F.R. § 152.25(g)(1)
Castor oil (U.S.P. or equivalent)
Cedar oil
Cinnamon and cinnamon oil
Citric acid
Citronella and citronella oil
Cloves and clove oil
Corn gluten meal
Corn oil
Cottonseed oil
Dried blood
Eugenol
Garlic and garlic oil
Geraniol
Geranium oil
Lauryl sulfate
Lemongrass oil
Linseed oil
Malic acid
Mint and mint oil
Peppermint and peppermint oil
2-Phenethyl propionate (2-phenylethyl propionate)
Potassium sorbate
Putrescent whole egg solids
Rosemary and rosemary oil
Sesame (includes ground sesame plant) and sesame oil
Sodium chloride (common salt)
Sodium lauryl sulfate
Soybean oil
Thyme and thyme oil
White pepper
Zinc metal strips (consisting solely of zinc metal and impurities)

Preferred FIFRA-exempt substances are essential oils, while more preferred FIFRA-exempt substances are cedar or cedarwood oil (i.e., Atlas, Himalayan, Texas, Virginian cedarwood oil), cinnamon oil, lemongrass oil, mint oil (i.e., corn mint, peppermint, spearmint oil), and mixtures thereof. These more preferred essential oils may be obtained from Kobashi Essential Oils, Kobashi 2 Fore Street, Ide, Devon EX2 9RQ, United Kingdom (“Kobashi Essential Oils”).

In addition to being exempt from FIFRA regulations, cedarwood oil and mint oil enjoy the additional benefit of avoiding further regulation by the International Fragrance Research Association (I.F.R.A.), when used at any level on or within materials/products intended for direct contact with skin, due to the recognized non-irritating nature of these oils.

The method of the present invention involves coating materials with one or more FIFRA-exempt substances and/or incorporating these substances within the material.

Materials contemplated for use with the inventive method are not limited. In several embodiments, which are described below, the material is in the form of a sheet or mat selected from the group of organic polymer sheets, elastomeric sheets (e.g., natural rubber latex, nitrile rubber latex (carboxylated or non-carboxylated), polychloroprene latex, polyisoprene latex, polyurethane latex), plastic sheets (e.g., polyethylene, polyethylene terephthalate (PET), polypropylene, polystyrene, and polyvinyl chloride plastic sheets), fibrous sheets or mats made of natural and/or synthetic (e.g., nylon, rayon) fibers, paper sheets, cellophane sheets, nylon sheets, rayon sheets and wax sheets. The material in these embodiments may be a single- or multi-layered construction.

The FIFRA-exempt substance(s) may be added to these sheets or mats either prior to, during, or post manufacture. More specifically, the FIFRA-exempt substance or substances may be incorporated within the starting material or materials prior to manufacture, or may be blended with the starting material(s) at the beginning of the manufacturing process. These substances may also be applied to and/or incorporated within the sheet or mat at a later stage in the manufacturing process or post manufacture.

By way of example, the FIFRA-exempt substance(s) may be compounded with a polymeric material (either alone or as applied to a carrier material) and the resulting composition extruded as sheets, films or fibers. The fibers may then be used to make a woven or non-woven sheet material or fabric.

By way of further example, the FIFRA-exempt substance(s) may be added later in a conventional paper-making process, for example, at the size press of a papermaking machine, or post manufacture. The FIFRA-exempt substance(s) may be added alone, as applied to a carrier material, or as part of a coating composition.

The phrase “carrier material”, as used herein, is intended to mean a porous carrier material that serves to immobilize the FIFRA-exempt substance(s) by adsorption, absorption or the like. By way of their porous structure, the carrier materials allow for increases in the FIFRA-exempt substance loadings within the natural or synthetic non-woven materials or fabrics. Moreover, the porous carrier material will serve to check or delay the release of these substances from the material, increasing the material's useful life.

Suitable porous carrier materials include, but are not limited to, kenaf fibers, siliceous minerals such as natural or synthetic clays (bentonite, attapulgite, fuller's earth, sepiolite, kaolin), silicates, synthetic porous silicas, talc, zeolite molecular sieves, and mixtures thereof.

Suitable methods or techniques for applying the FIFRA-exempt substances to sheets or mats either during or post manufacture include, but are not limited to, immersion, dipping, spraying, misting, foaming, and fogging or vapor bath. The FIFRA-exempt substance(s) may also be provided on a roller or brush which contacts one or more surfaces of the material.

As will be readily appreciated by those skilled in the art, non-porous materials subjected to one of the above-identified application methods will have one or more FIFRA-exempt substances coated on at least one surface, while porous materials will have the FIFRA-exempt substance(s) contained on and within the material.

The FIFRA-exempt substances may also be incorporated within an adhesive or glue used to laminate sheets or mats together to form a multi-layered structure. In one such contemplated embodiment, the inventive material is a multi-layered structure comprising two or more plastic sheets (e.g., polyethylene, PET, polypropylene, and polystyrene plastic sheets) laminated together using an isocyanate (NCO)-containing laminating adhesive that contains an effective amount of one or more FIFRA-exempt substances. Suitable NCO-containing laminating adhesives are available from Henkel Adhesives, 1345 Gasket Drive, Elgin, Ill. 60120, under the product designation LIOFOL NCO-containing laminating adhesives. In a preferred embodiment, PET and polyethylene films or plastic sheets are used to prepare the inventive multi-layered structure and the laminating adhesive contains at least about 50% by wt. of one or more FIFRA-exempt substances. In another preferred embodiment, a non-porous or barrier film (e.g., PET films and polyvinylidene dichloride (PVdC)-coated PET films) and a porous or selectively permeable film (e.g., low density polyethylene (LDPE) films and films prepared from blends of LDPE and cyclic olefins) are used to prepare the inventive structure. The non-porous or barrier film would serve to prevent migration of the FIFRA-exempt substance(s) through the barrier film, while the porous or selectively permeable film would allow for controlled release of the FIFRA-exempt substance(s) to an outer surface thereof. The inventive multi-layered structures may be used to manufacture, for example, bags and lids for containers or vacuum packaging.

The FIFRA-exempt substances may also be applied to a surface of a first sheet or mat material, covering either the entire surface or only select areas or pockets, and a second sheet or mat material applied over the surface of the first material and the material sheets sealed together using a laminating adhesive so as to trap the substance(s) between the sheets. In a preferred embodiment, the sheets are sealed together in a pattern so that a pattern of pockets or cells are formed containing the FIFRA-exempt substance or substances.

In another embodiment contemplated by way of the present invention, the material is in the form of a solvent- or water-based paint or paint formulation, while in yet another embodiment, the material is in the form of a wax-based composition.

Products exhibiting antimicrobial, insecticidal, biocidal and/or odor controlling properties that are prepared using the inventive materials include (in addition to those noted above), but are not limited to, protective clothing such as gloves, coveralls, face masks, hats, lab coats and isolation gowns, medical and dental protective barriers, protective footwear, footwear liners, groundcover for agricultural (e.g., row covers, plastic mulching), plastic food wraps, food packaging liners, fresh food case liners, wax paper, wax boxes, wax wraps, coated food items such as coated fruits and vegetables, garbage bags, food labels, cut flower wraps, air filters, STYROFOAM packaging, floor mats, candles, and painted items.

In one preferred embodiment, the inventive product is a flexible, protective glove having an inside surface that is coated with one or more FIFRA-exempt substances, preferably one or more FIFRA-exempt essential oils. The inventive gloves are antimicrobial and control airborne pathogens and bacteria colonies, and thus are suitable for use by medical doctors, dentists, health care workers, food handlers, and the like.

Preferred flexible materials for use in preparing the inventive gloves take the form of a non-porous, synthetic non-woven material or plastic sheet having a thickness ranging from about 1 to about 10 millimeters (mm) (preferably, from about 2 to about 5 mm).

Preferred FIFRA-exempt essential oils for these skin-contact products are cedarwood oil and mint oil. For those gloves employing cedarwood oil, the oil is preferably applied to a surface of the material at a rate of from about 1 to about 30 milliliters (mils) per square foot (more preferably, at a rate of from about 15 to about 25 mils per square foot), while for those gloves employing mint oil, the oil is preferably applied to a surface of the material at a rate of from about 1 to about 10 mils per square foot (more preferably, at a rate of from about 3 to about 7 mils per square foot).

In another preferred embodiment, the inventive product is a row cover used to shield plants from frost, while protecting the plants from insects, and the bacteria, molds and fungus in the soil. These products are preferably prepared from optionally colored plastic sheet materials (e.g., spun polypropylene sheet materials) that are coated and/or impregnated with one or more FIFRA-exempt substances, preferably one or more FIFRA-exempt essential oils.

In cold areas, such as New England, growers place black plastic sheets over the soil. These absorb the sun's heat, allowing the soil to be planted sooner. The seedlings develop quickly in the warmer soil. The plastic is removed once the seedlings are well-established.

More preferred materials for use in preparing the inventive row covers have a basis weight ranging from about 1 to about 50 grams per square meter (g/m²) (preferably, from about 2 to about 5 g/m²), and a thickness ranging from about 1 to about 10 mm (preferably, from about 2 to about 5 mm).

Preferred FIFRA-exempt essential oils for these row covers are cedar or cedarwood oil, cinnamon oil, lemongrass oil, mint oil, and mixtures thereof. Also preferred for use with row covers is FIFRA-exempt potassium sorbate.

In yet another preferred embodiment, the inventive product is a painted item having at least one surface covered by a paint or paint formulation having at least one FIFRA-exempt substance incorporated therein. Paints contemplated by the present invention include both solvent- and water-based paints, which typically include a film-forming binder component and a color-producing component. The inventive paint protects painted surfaces by killing mold, fungus, bacteria and insects that come in contact with the paint.

In a preferred embodiment, the paint or paint formulation is prepared using solvent-based paint and an effective amount (i.e., 1 to 3% by wt.) of a FIFRA-exempt essential oil.

In yet another preferred embodiment, the inventive product is prepared from (or coated with) a wax-based composition having at least one FIFRA-exempt substance incorporated therein.

Examples of waxes suitable for use in preparing the wax-based composition include, but are not limited to, carnauba wax, candelilla wax, beeswax, synthetic wax, shellac wax, spermaceti, lanolin wax, ozokerite, bran wax, ceresin wax, bayberry wax, paraffin, rice wax and jojoba wax.

In a preferred embodiment, the wax-based composition is prepared using paraffin wax and an effective amount (i.e., 1 to 10% by wt.) of cedar or cedarwood oil, cinnamon oil, lemongrass oil and/or mint oil.

The wax-based composition may be formed into candles or may be applied directly to the surface of, for example, produce (e.g., fruits and vegetables), paper, cardboard, fabrics, and the like.

### WORKING EXAMPLE

A quantity of one hundred (100) test samples were prepared by applying a 50/50 mixture of Virginian cedarwood and peppermint oils (obtained from Kobashi Essential Oils) to the inside surface of fifty (50) vinyl medical examination gloves and fifty (50) latex medical examination gloves (vinyl and latex medical examination gloves obtained from Patterson Dental Supply, Inc., 1031 Mendota Heights Road, St. Paul, Minn. 55120) at a rate of 5 mils/ft². The test samples were air dried and then a 2.54 centimeter (cm)×2.54 cm patch was cut from the center of the palm portion of each glove.

A quantity of twenty (20) control samples were prepared by cutting identically sized patches from the center of the palm portion of ten (10) uncoated vinyl medical examination gloves and ten (10) uncoated latex medical examination gloves, which were obtained from the same source.

B. Subtilis bacteria (obtained from Nelson Laboratories, Inc., 6280 South Redwood Road, Salt Lake City, Utah 84123-6600) was uniformly swabbed onto one hundred and twenty (120) Tryptic Soy Agar (T.S.A.) dishes. Each test sample and control patch was then placed (inside surface facing down) in the center of an inoculated agar dish. The dishes were kept at 21° C. for seventy-two (72) hours.

The diameter of each zone (including the diameter of the patch) was measured and recorded in centimeters. The average zone of inhibition for the test samples was 0.5 cm, with no growth of bacteria observed under any test sample. The control samples or patches did not exhibit a zone of inhibition, with growth of bacteria observed up to the edge of (and under) each patch.

Although the present invention has been shown and described with respect to detailed embodiments thereof, it will be understood by those skilled in the art that various changes in form and detail thereof may be made without departing from the spirit and scope of the claimed invention.

Having thus described the invention, what is claimed is:

1. A method for using one or more FIFRA-exempt substances to provide a material with antimicrobial, insecticidal, biocidal and/or odor controlling properties, which comprises applying or adding one or more FIFRA-exempt substances to the material.

2. The method of claim 1, wherein the material is in the form of a sheet or mat selected from the group of organic polymer sheets, elastomeric sheets, plastic sheets, fibrous sheets or mats made of natural and/or synthetic fibers, paper sheets, cellophane sheets, nylon sheets, rayon sheets and wax sheets.

3. The method of claim 1, wherein the material is in the form of a paint or paint formulation.

4. The method of claim 1, wherein the material is in the form of a wax-based composition.

5. The method of claim 1, wherein the one or more FIFRA-exempt substances are selected from the group of castor oil, cedar or cedarwood oil, cinnamon and cinnamon oil, citric acid, citronella and citronella oil, cloves and clove oil, corn gluten meal, corn oil, cottonseed oil, dried blood, eugenol, garlic and garlic oil, geraniol, geranium oil, lauryl sulfate, lemongrass oil, linseed oil, malic acid, mint and mint oil, peppermint and peppermint oil, 2-phenylethyl propionate, potassium sorbate, putrescent whole egg solids, rosemary and rosemary oil, sesame and sesame oil, sodium chloride, sodium lauryl sulfate, soybean oil, thyme and thyme oil, white pepper, zinc metal strips, and mixtures thereof.

6. The method of claim 5, wherein the one or more FIFRA-exempt substances are selected from the group of cedar or cedarwood oil, cinnamon oil, lemongrass oil, mint oil, and mixtures thereof.

7. A material in the form of a sheet or mat, a paint or paint formulation, or a wax-based composition that exhibits antimicrobial, insecticidal, biocidal and/or odor controlling properties, and that comprises one or more FIFRA-exempt substances.

8. The material of claim 7, wherein the material is in the form of a sheet or mat selected from the group of organic polymer sheets, elastomeric sheets, plastic sheets, fibrous sheets or mats made of natural and/or synthetic fibers, paper sheets, cellophane sheets, nylon sheets, rayon sheets and wax sheets.

9. The material of claim 8, wherein the sheet or mat is a plastic sheet selected from the group of polyethylene, polyethylene terephthalate, polypropylene, polystyrene, and polyvinyl chloride plastic sheets.

10. The material of claim 9, which is a multi-layered structure that comprises two or more plastic sheets laminated together using an adhesive containing an effective amount of one or more FIFRA-exempt substances.

11. The multi-layered structure of claim 10, which comprises a polyethylene terephthalate plastic sheet laminated to a polyethylene plastic sheet with an isocyanate-containing laminating adhesive that contains an effective amount of one or more FIFRA-exempt substances.

12. The multi-layered structure of claim 10, which comprises a non-porous or barrier film laminated to a porous or selectively permeable film using a laminating adhesive that contains an effective amount of one or more FIFRA-exempt substances.

13. The material of claim 7, wherein the material is in the form of a paint or paint formulation.

14. The material of claim 7, wherein the material is in the form of a wax-based composition.

15. The material of claim 14, wherein the wax of the wax-based composition is selected from the group of carnauba wax, candelilla wax, beeswax, synthetic wax, shellac wax, spermaceti, lanolin wax, ozokerite, bran wax, ceresin wax, bayberry wax, paraffin, rice wax, jojoba wax, and combinations thereof.

16. The material of claim 7, wherein the one or more FIFRA-exempt substances are selected from the group of castor oil, cedar or cedarwood oil, cinnamon and cinnamon oil, citric acid, citronella and citronella oil, cloves and clove oil, corn gluten meal, corn oil, cottonseed oil, dried blood, eugenol, garlic and garlic oil, geraniol, geranium oil, lauryl sulfate, lemongrass oil, linseed oil, malic acid, mint and mint oil, peppermint and peppermint oil, 2-phenylethyl propionate, potassium sorbate, putrescent whole egg solids, rosemary and rosemary oil, sesame and sesame oil, sodium chloride, sodium lauryl sulfate, soybean oil, thyme and thyme oil, white pepper, zinc metal strips, and mixtures thereof.

17. The material of claim 16, wherein the one or more FIFRA-exempt substances are selected from the group of cedar or cedarwood oil, cinnamon oil, lemongrass oil, mint oil, and mixtures thereof.

18. A flexible protective glove that exhibits antimicrobial, insecticidal, biocidal and/or odor controlling properties and that comprises: a glove body having an inside surface and an outside surface; and one or more FIFRA-exempt substances coated on at least the inside surface of the glove body.

19. A row cover for shielding and protecting plants that exhibits antimicrobial, insecticidal, biocidal and/or odor controlling properties and that comprises a plastic sheet material that is coated and/or impregnated with one or more FIFRA-exempt substances.
